(19) United States
(12) Patent Application Publication
(10) Pub. No.: US 2024/0384215 A1
(43) Pub. Date: Nov. 21, 2024

(54) CELL CULTURING DEVICE

(71) Applicant: SHIMADZU CORPORATION, Kyoto-shi, Kyoto (JP)

(72) Inventor: Tomohisa HASUNUMA, Kobe-shi, Hyogo (JP)

(73) Assignee: SHIMADZU CORPORATION, Kyoto-shi, Kyoto (JP)

(21) Appl. No.: 18/687,653

(22) PCT Filed: Feb. 18, 2022
(86) PCT No.: PCT/JP2022/006510

§ 371 Date: May 24, 2024

(30) Foreign Application Priority Data
Aug. 31, 2021 (JP) 2021-141477

(51) Int. Cl. C12M 1/06; C12M 1/00; C12M 1/34

(52) U.S. Cl. CPC C12M 27/02; C12M 29/00; C12M 41/42

(57) ABSTRACT

The cell culturing device includes a vessel that accommodates a culture solution containing a medium and cells, an agitator that is provided in the vessel and agitates the culture solution, a discharge pipe that discharges the culture solution in the vessel to outside the vessel, and an oxygen intake pipe that draws oxygen into the vessel. The discharge pipe has an end below the agitator in the vertical direction.

### TECHNICAL FIELD

The present disclosure relates to a cell culturing device.

### BACKGROUND ART

As disclosed in PTL 1, there is known a device for culturing cells such as microorganisms by adjusting a dissolved oxygen concentration while agitating a culture solution in a vessel.

### CITATION LIST

### Patent Literature

PTL 1: WO 2020/017407

### SUMMARY OF INVENTION

### Technical Problem

The device disclosed in PTL 1 has a possibility of inaccurately aspirating and dispensing a sample as a proportion of gas in an aspirated culture solution varies due to an effect of oxygen bubbling caused by supplying oxygen.

The present disclosure has been made to solve such a problem, and an object of the present disclosure is to provide a cell culturing device capable of accurately aspirating and dispensing a sample while maintaining a constant amount of gas dissolved in a culture solution.

### Solution to Problem

The present disclosure relates to a cell culturing device for culturing cells. The cell culturing device comprises a vessel that accommodates a culture solution containing a medium and cells, an agitator that is provided in the vessel and agitates the culture solution, a discharge pipe that discharges the culture solution in the vessel to outside the vessel, and an oxygen intake pipe that draws oxygen into the vessel. The discharge pipe has an end below the agitator in a vertical direction.

### Advantageous Effects of Invention

According to the present disclosure, a sample can be accurately aspirated and dispensed while a constant amount of gas dissolved in a culture solution is maintained.

### BRIEF DESCRIPTION OF DRAWINGS

FIG. 1 is a block diagram showing a schematic configuration of an automated pretreatment system.

FIG. 2 is a channel diagram showing a channel configuration of a sampling apparatus.

FIG. 3 is a block diagram showing a schematic configuration of a control device.

FIG. 4 is a perspective view of a cell culturing device.

FIG. 5 is a plan view of the cell culturing device having some components removed therefrom.

FIG. 6 is a partial cross section taken along a line VI-VI indicated in FIG. 5.

FIG. 7 is a partial cross section taken along a line VII-VII indicated in FIG. 5.

FIG. 8 is a diagram showing an internal structure of the cell culturing device.

FIG. 9 shows an agitator.

FIG. 10 shows a state with a shaft unit having the agitator removed therefrom.

FIG. 11 is a flowchart of a process performed in a sampling apparatus 1 to sample a culture solution in a cell culturing device 100 and introduce the sampled culture solution into a test tube 14.

FIG. 12 is a diagram showing an example of a result of an amount of a liquid introduced according to the process of FIG. 11.

FIG. 13 is a diagram showing an example of a result of an amount of a liquid introduced according to a comparative example.

### DESCRIPTION OF EMBODIMENTS

An embodiment will now be described in detail with reference to the drawings. In the figures, identical or corresponding components are identically denoted and in principle will not be described repeatedly.

### Schematic Configuration of Automated Pretreatment System

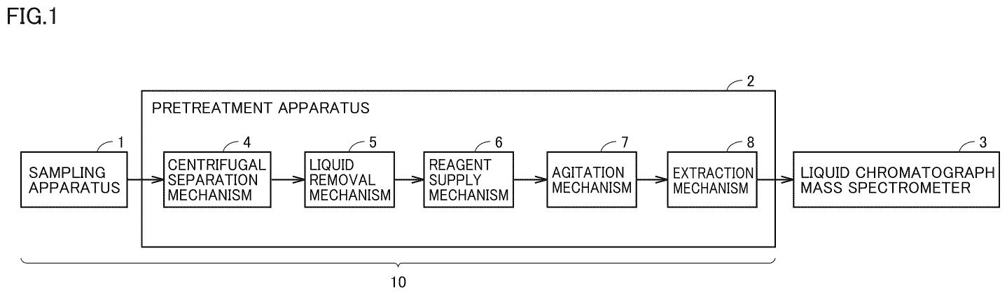

FIG. 1 is a block diagram showing a schematic configuration of an automated pretreatment system 10. Automated pretreatment system 10 is an apparatus for automatically subjecting an analyte to a pretreatment. In the present embodiment, the analyte is for example a cultured cell, and more specifically, it is a bacterial cell.

Automated pretreatment system 10 comprises a sampling apparatus 1 and a pretreatment apparatus 2. Metabolites are extracted from cells after automated pretreatment system 10 subjects the cells to the pretreatment. The extracted metabolites are supplied to a liquid chromatograph mass spectrometer 3. Liquid chromatograph mass spectrometer 3 is only an example of an analyzer for analyzing an analyte. Other analyzers can also be used to analyze the analyte.

Sampling apparatus 1 is an apparatus for sampling a liquid from a vessel (a culture vessel). For example, cells of microorganisms, plants and the like are cultured in a culture solution containing a medium in a vessel called a bioreactor. The bioreactor is provided therein for example with an agitating member rotated using magnetic force, an oxygen concentration sensor for sensing a dissolved oxygen concentration, and the like. Cells are cultured in sampling apparatus 1 by adjusting a dissolved oxygen concentration while agitating the culture solution containing the medium and the cells in the bioreactor. A detailed description of the bioreactor functioning as a cell culturing device will be given later.

Pretreatment apparatus 2 subjects cells contained in the culture solution sampled from an interior of the bioreactor (i.e., a culture sample) to pretreatment. In sampling apparatus 1, a culture solution containing cells is stored in a test tube serving as a vessel (a sampling vessel). Pretreatment apparatus 2 comprises a centrifugal separation mechanism 4, a liquid removal mechanism 5, a reagent supply mechanism 6, an agitation mechanism 7, an extraction mechanism 8, and the like. These mechanisms sequentially subject to pretreatment the cells contained in the culture solution in the test tube.

Centrifugal separation mechanism 4 applies centrifugal force to the culture solution in the test tube. Thereby, the culture solution in the test tube is separated, with a solid-liquid interface as a boundary, into a solid component that sediments on the bottom of the test tube and a liquid component that floats above the solid component. The solid component is a culture, e.g., cultured cells. The liquid component floating on the solid component is supernatant separated from the culture solution.

Liquid removal mechanism 5 aspirates the supernatant from the test tube. This removes the liquid in the test tube and the cells remain in the test tube. Reagent supply mechanism 6 supplies the cells in the test tube with a reagent for extracting metabolites in the cells. This produces a liquid of a mixture of the cells and the reagent in the test tube. Agitation mechanism 7 agitates the mixture liquid. By agitating the mixture liquid, a suspension in which metabolites are extracted from the cells is obtained.

Extraction mechanism 8 extracts a portion of the suspension as an extract. The extract is supplied to liquid chromatograph mass spectrometer 3.

### Schematic Configuration of Sampling Apparatus

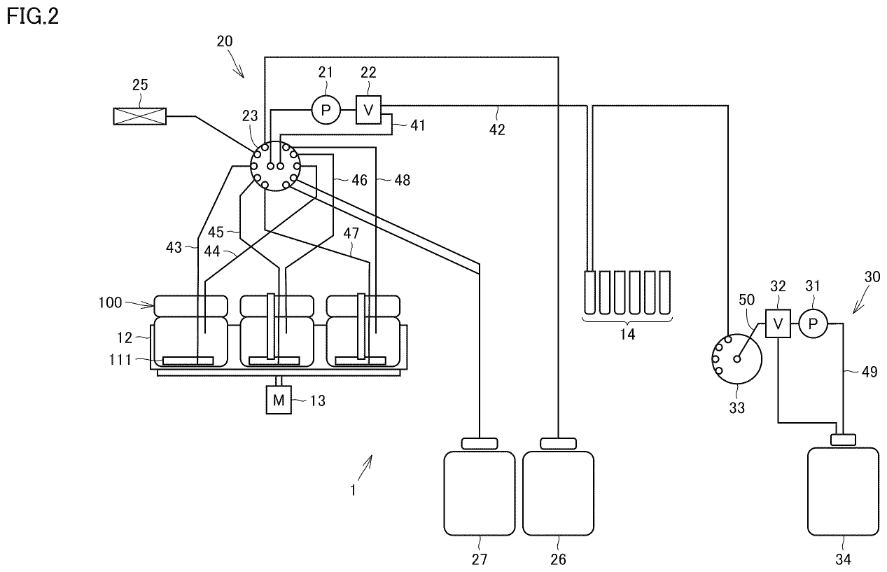

FIG. 2 is a channel diagram showing a channel configuration of sampling apparatus 1. Sampling apparatus 1 samples a culture solution containing cells in cell culturing device 100 referred to as a bioreactor. Cell culturing device 100 comprises an agitator 111 as an agitating member rotated using magnetic force.

Cell culturing device 100 is held by a holding unit 12 provided in sampling apparatus 1. In the present embodiment, one holding unit 12 can hold three cell culturing devices 100, and a plurality of (for example, four) such holding units 12 are provided. Only a single holding unit 12 may be provided. Holding unit 12 may be configured to hold two or less or four or more cell culturing devices 100.

Cell culturing device 100 can perform culturing while it is heated by a heater (not shown) provided in holding unit 12. A motor 13 for rotating a magnet (not shown) is connected to holding unit 12. Rotating motor 13 rotates the magnet, and the magnet's magnetic force can rotate agitator 111 in each cell culturing device 100.

Sampling apparatus 1 can perform culturing by agitating the culture solution by agitator 111 while controlling the temperature of the culture solution in cell culturing device 100. Sampling apparatus 1 samples the culture solution containing cultured cells and introduces the sampled culture solution into test tube 14 at any desired time.

Sampling apparatus 1 comprises a culture solution sampling mechanism 20 for sampling and introducing a culture solution into test tube 14, and a reagent sampling mechanism 30 for sampling and introducing a reagent into test tube 14. Test tube 14 receives a mixture solution obtained by mixing the culture solution and the reagent together, and is sealed by a cap (not shown) and thus transferred to pretreatment apparatus 2.

Culture solution sampling mechanism 20 is provided with a pump 21 and a plurality of valves 22 and 23. Valve 23 for example has a pair of common ports and five pairs of selective ports (ten in total), and a channel can be switched by selecting any one pair of selective ports, as desired, and connecting the selected pair of selective ports to the pair of common ports.

Pump 21 and valve 22 are provided along a channel 41 interconnecting the paired common ports. Valve 22 constitutes a channel switching unit (a first channel switching unit) for switching whether a liquid in channel 41 is guided to a branch channel 42 branched with respect to channel 41. That is, valve 22 can switch between a state in which a liquid is caused to communicate between the paired common ports via channel 41 and a state in which the liquid in channel 41 is guided to branch channel 42.

Of the five pairs of selective ports, one pair of selective ports is connected to an extraction channel 43 and an introduction channel 44, respectively, that communicate with one cell culturing device 100. Extraction channel 43 is a channel for extracting the culture solution in cell culturing device 100. On the other hand, introduction channel 44 is a channel for re-introducing into cell culturing device 100 the culture solution extracted from cell culturing device 100 through extraction channel 43 and circulating through channel 41. Another pair of selective ports is connected to an extraction channel 45 and an introduction channel 46, respectively, that communicate with another cell culturing device 100. Still another pair of selective ports is connected to an extraction channel 47 and an introduction channel 48 that communicate with still another cell culturing device 100.

Sampling apparatus 1 allows any one of extraction channels 43, 45, 47 and a corresponding one of introduction channels 44, 46, 48 to communicate with each other through channel 41 and allows pump 21 to be driven in that state to circulate the culture solution in each cell culturing device 100. That is, channel 41, extraction channels 43, 45, 47, and introduction channels 44, 46, 48 constitute a circulation channel for circulating the culture solution in each cell culturing device 100 (i.e., a first circulation channel).

Pump 21 pumps out the culture solution from each cell culturing device 100 into the first circulation channel and introduces the culture solution from the first circulation channel into each cell culturing device 100 to constitute a circulation mechanism (a first circulation mechanism) for causing the culture solution in each cell culturing device 100 to circulate through the first circulation channel.

Extraction channels 43, 45, 47 each have a distal end immersed in the culture solution in the corresponding cell culturing device 100. In contrast, introduction channels 44, 46, 48 each have a distal end spaced upward from the culture solution in the corresponding cell culturing device 100. The culture solution extracted from cell culturing device 100 through each extraction channel 43, 45, 47 and circulated through channel 41 falls from the distal end of each introduction channel 44, 46, 48 and is introduced into cell culturing device 100.

In sampling apparatus 1, at least a portion of channel 41 interconnecting the paired common ports that is provided with pump 21 is formed of a flexible tube. Pump 21 is for example a tubing pump and allows the flexible tube to be deformed (or compressed and relaxed) to deliver a liquid internal to the tube.

By switching valve 22 serving as the first channel switching unit provided at an intermediate portion of channel 41, the culture solution circulating into each cell culturing device 100 through channel 41 can be flowed out to branch channel 42. In doing so, branch channel 42 has a distal end disposed in test tube 14, and the culture solution is sampled and introduced into test tube 14 through branch channel 42.

Other than the three pairs of selective ports to which extraction channels 43, 45, 47 and introduction channels 44, 46, 48 are connected, two pairs of selective ports has one pair of selective ports connected to a cleaning liquid tank 26 and a waste liquid tank 27, respectively. The remaining one pair of selective ports is connected to a filter 25 and waste liquid tank 27, respectively. Cleaning liquid tank 26 stores a cleaning liquid for cleaning the channels for the culture solution.

After the culture solution is sampled from any of cell culturing devices 100 and introduced into test tube 14, valve 23 is switched to connect cleaning liquid tank 26 and waste liquid tank 27 to channel 41, and when pump 21 is driven in that condition, the cleaning liquid in cleaning liquid tank 26 is discharged through channel 41 into waste liquid tank 27. Channel 41, valve 22, and the like can thus be cleaned with the cleaning liquid.

After the cleaning with the cleaning liquid, valve 23 is switched to connect filter 25 and waste liquid tank 27 to channel 41, and when pump 21 is driven in that condition, air is introduced into channel 41 through filter 25 and discharged to waste liquid tank 27 together with moisture remaining in channel 41. This can remove moisture from channel 41, valve 22, and the like.

Reagent sampling mechanism 30 is provided with a pump 31 and a plurality of valves 32 and 33. Valve 33 for example has one common port and a plurality of selective ports, and can switch a channel by selecting one of the selective ports, as desired, and connecting the selected port to the common port.

Pump 31 and valve 32 are provided along a channel 49 having opposite ends in communication with a reagent tank 34. Reagent tank 34 contains a reagent to be mixed with the culture solution sampled and introduced in test tube 14. Channel 49 constitutes a circulation channel (a second circulation channel) for circulating the reagent in reagent tank 34. Pump 31 pumps out the reagent from reagent tank 34 into the second circulation channel and introduces the reagent from the second circulation channel into reagent tank 34 to constitute a circulation mechanism (a second circulation mechanism) to allow the reagent in reagent tank 34 to circulate through the second circulation channel.

In sampling apparatus 1, at least a portion of channel 49 having opposite ends connected to reagent tank 34 that is provided with pump 31 is formed of a flexible tube. Pump 31 is for example a tubing pump and allows the flexible tube to be deformed (or compressed and relaxed) to deliver a liquid internal to the tube.

Valve 32 constitutes a channel switching unit (a second channel switching unit) for switching whether a liquid in channel 49 should be guided to a branch channel 50 branched with respect to channel 49. That is, valve 32 can switch between a state in which the reagent in reagent tank 34 is circulated through channel 49 and a state in which the reagent in channel 49 is guided to branch channel 50.

By thus switching valve 32 serving as the second channel switching unit provided at an intermediate portion of channel 49, the reagent circulating into reagent tank 34 through channel 49 can be caused to flow out to branch channel 50. Branch channel 50 is connected to the common port of valve 33, and any one of the selective ports of valve 33 is connected to test tube 14. Therefore, by causing the selective port connected to test tube 14 to communicate with the common port, the reagent flowing out from channel 49 to branch channel 50 can be sampled and introduced into test tube 14.

### Schematic Configuration of Control Device

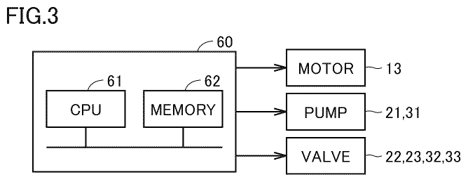

FIG. 3 is a block diagram showing a schematic configuration of control device 60. Sampling apparatus 1 comprises control device 60. Control device 60 for example includes a CPU (Central Processing Unit) 61 and a memory 62. Memory 62 is for example composed of a ROM (Read Only Memory) and a RAM (Random Access Memory) and can store a control program and a variety of data. CPU 61 can control operations of motor 13, pumps 21 and 31, valves 22, 23, 32 and 33, and the like by executing the control program stored in memory 62.

Control device 60 can circulate the culture solution in any one of cell culturing devices 100 by driving pump 21 at a constant liquid pumping rate while any one of extraction channels 43, 45, 47 communicates with a corresponding one of introduction channels 44, 46, 48 through channel 41. Control device 60 can sample the culture solution in channel 41 and introduce the sampled culture solution into test tube 14 by switching valve 22 for a predetermined period of time based on the control program to cause channel 41 to communicate with branch channel 42.

Control device 60 can control an amount of the culture solution to be sampled by controlling when a channel is switched by valve 22. That is, if the liquid pumping rate of pump 21 is known in advance, a desired amount of the culture solution can be accurately sampled and introduced into test tube 14 by adjusting a period of time for which channel 41 is caused to communicate with branch channel 42.

Control device 60 can circulate the reagent in reagent tank 34 by driving pump 31 at a constant liquid pumping rate while channel 49 is in communication from its one to other ends. Control device 60 can sample the reagent in channel 49 and introduce the sampled regent into test tube 14 by switching valve 32 for a predetermined period of time based on the control program to cause channel 49 to communicate with branch channel 50, and also switching valve 33 to cause branch channel 50 to communicate with test tube 14.

Control device 60 can control an amount of the reagent to be sampled by controlling when a channel is switched by valve 32. That is, if the liquid pumping rate of pump 31 is known in advance, a desired amount of the reagent can be accurately sampled and introduced into test tube 14 by adjusting a period of time for which channel 49 is caused to communicate with branch channel 50.

### Configuration of Cell Culturing Device

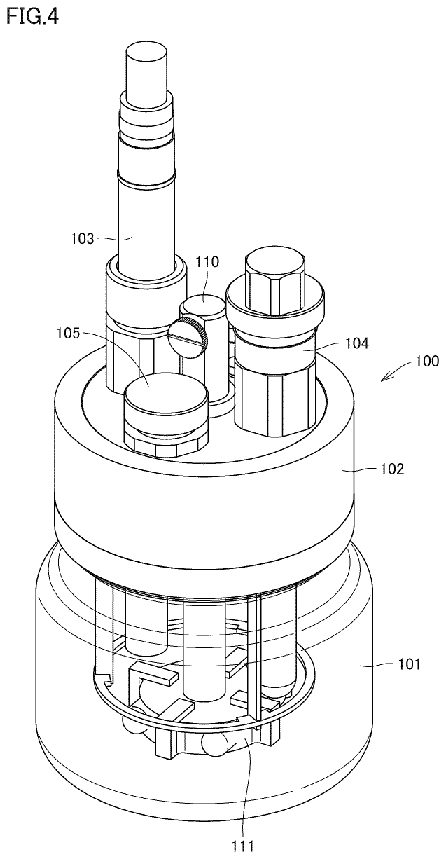

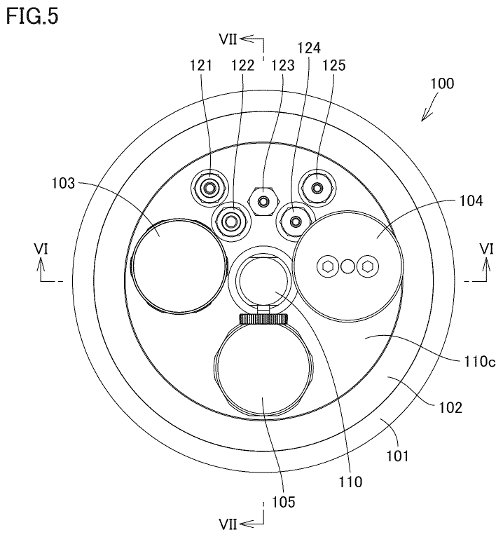

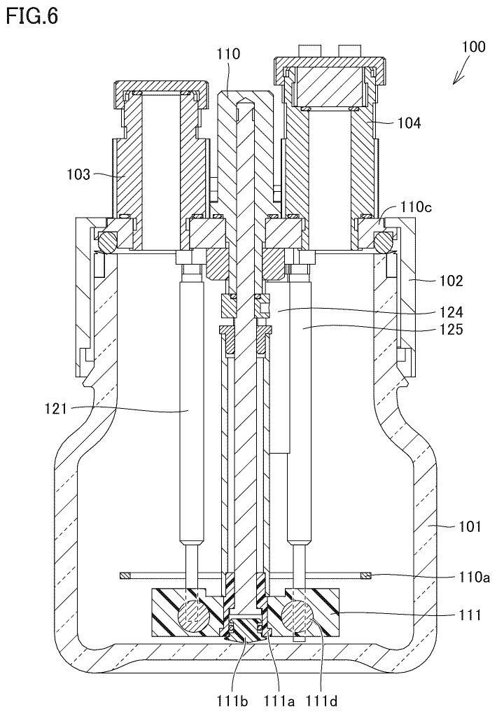

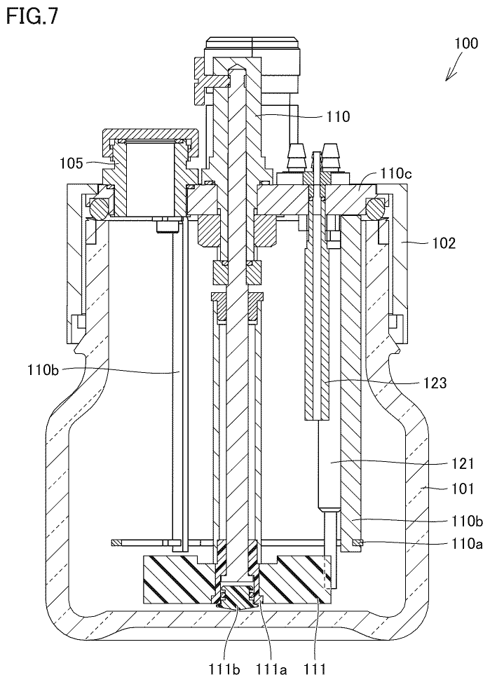

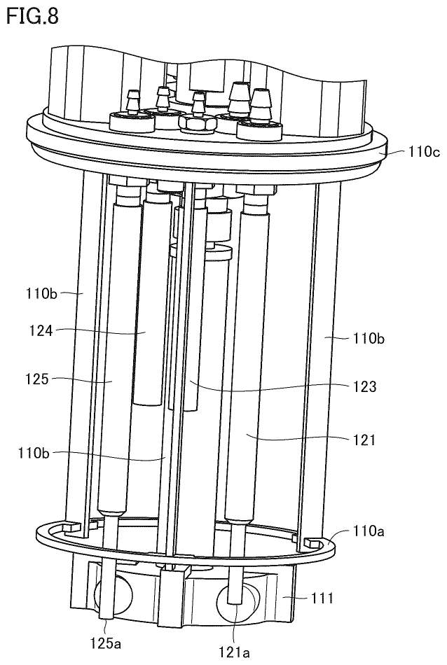

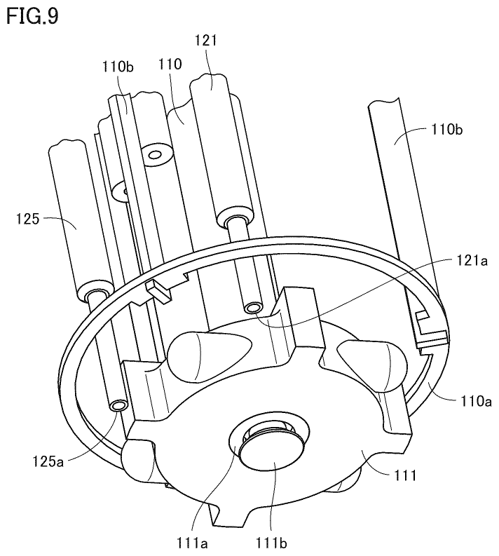

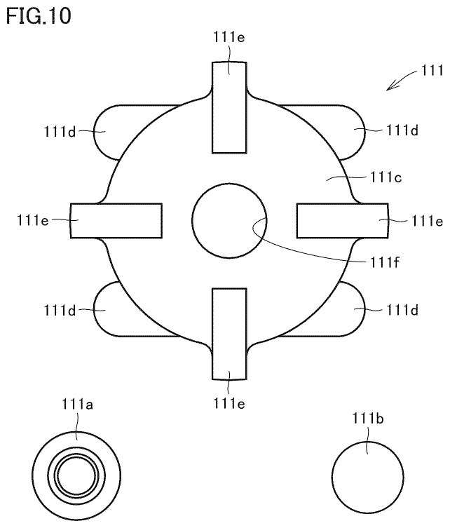

With reference to FIGS. 4 to 10, a structure of cell culturing device 100 serving as a cell culturing device will be described. FIG. 4 is a perspective view of cell culturing device 100, FIG. 5 is a plan view showing cell culturing device 100 having some components removed therefrom, FIG. 6 is a partial cross section taken along a line VI-VI indicated in FIG. 5, FIG. 7 is a partial cross section taken along a line VII-VII indicated in FIG. 5, FIG. 8 is a view showing an internal structure of cell culturing device 100, FIG. 9 shows agitator 111, and FIG. 10 shows a state with a shaft unit 110 having agitator 111 removed therefrom.

As shown in FIG. 4, cell culturing device 100 includes a vessel 101, a lid unit 102, a DO (Dissolved Oxygen) sensor 103 connected to lid unit 102, a pH sensor 104, a cap 105, and shaft unit 110.

Vessel 101 is a transparent vessel receiving a culture solution containing microorganisms, plant cells, and the like. Lid unit 102 is for sealing vessel 101 and has a variety of components attached thereto. DO sensor 103 is a sensor for measuring a dissolved oxygen concentration in cell culturing device 100. PH sensor 104 is a sensor for measuring a concentration of hydrogen ions in the culture solution. Cap 105 is a lid for an opening projecting at an upper portion of cell culturing device 100. Shaft unit 110 is a member serving as a shaft for agitator 111 and allowing agitator 111 attached to an end thereof to rotate.

FIGS. 5 to 7 show a state with DO sensor 103 and pH sensor 104 having some components removed therefrom. In the plan view of FIG. 5, five pipes connected to a flexible tube are provided between DO sensor 103 and pH sensor 104. The five pipes include an oxygen intake pipe 121, an oxygen exhaust pipe 122, a sample adding pipe 123, a suction pipe 124, and a discharge pipe 125.

Oxygen intake pipe 121 is a pipe for supplying a culture solution with oxygen and, as shown in FIGS. 6 and 7, extends to agitator 111 located at a lower position in vessel 101. Oxygen exhaust pipe 122 is a pipe for exhausting excess oxygen from cell culturing device 100. Sample adding pipe 123 is a pipe for adding a sample as necessary. Suction pipe 124 is a pipe connected to any one of introduction channels 44, 46 and 48 for introducing a culture solution into cell culturing device 100. Discharge pipe 125 is a pipe connected to any one of extraction channels 43, 45 and 47, for discharging the culture solution in cell culturing device 100 to outside of cell culturing device 100.

The five pipes each have a length, as described below. Oxygen exhaust pipe 122 is the shortest among the five pipes, and extends to a position overlapping lid unit 102 in a vertical direction (a downward direction of an upward/downward direction in the plane of the drawing of the figure). Sample adding pipe 123 and suction pipe 124 are substantially equal in length, and are longer than oxygen exhaust pipe 122 and extend to a center position of vessel 101 in the vertical direction.

Oxygen intake pipe 121 is longer than sample adding pipe 123 and suction pipe 124 and extends to a position overlapping agitator 111 in the vertical direction. Discharge pipe 125 is longer than oxygen intake pipe 121 and extends to a position below agitator 111 in the vertical direction.

The five pipes are fixed to a pedestal unit 110c on an upper surface of lid unit 102 together with shaft unit 110. Three baffle plates 110b extend downward from pedestal unit 110c as shown in FIG. 8. Baffle plate 110b has an end fixed to an annular component 110a. Of the five pipes, oxygen intake pipe 121 and discharge pipe 125 are fixed to annular component 110a.

Baffle plate 110b is a member for forming a turbulent flow that also generates a vertical flow with respect to a lateral flow generated as agitator 111 rotates. Agitator 111 has a magnet unit 111d with a magnet disposed therein. As shown in FIGS. 6 to 9, oxygen intake pipe 121 has an end 121a positioned so as to overlap agitator 111 in the vertical direction, and discharge pipe 125 has an end 125a positioned below agitator 111 in the vertical direction.

As described above, oxygen intake pipe 121 has end 121a positioned so as to overlap agitator 111 and discharge pipe 125 has end 125a positioned below agitator 111 so that when the culture solution is agitated by agitator 111 the culture solution can be discharged to outside of vessel 101 at a position hardly affected by oxygen bubbling caused by supplying oxygen. Cell culturing device 100 can thus accurately aspirate and dispense a sample while keeping a constant amount of gas dissolved in a culture solution.

Hereinafter, agitator 111 will be described in detail. As shown in FIG. 10, agitator 111 includes a body 111c, a bearing unit 111a, and a locking unit 111b. Body 111c is formed in a cylinder having a center provided with a hole 111f and has a rotary blade unit 111e formed at every 90°. Agitator 111 includes two magnet units 111d projecting from body 111c. Magnet unit 111d is coated with a material similar to that for body 111c.

Body 111c of agitator 111 is made of polyetheretherketone. Polyetheretherketone is generally abbreviated as PEEK, and accordingly, hereinafter it will be referred to as PEEK. Rotary blade unit 111e and magnet unit 111d formed integrally with body 111c are also coated with a material composed of PEEK. In contrast, bearing unit 111a and locking unit 111b are formed of polyacetal (abbreviation: POM).

Polyacetal and PEEK are both resin and have different properties. Polyacetal has an amorphous portion and a crystalline portion mixed together, and accordingly, is used as an engineering plastic excellent in strength, modulus of elasticity, and impact resistance. Polyacetal is also used as a bearing component because of its excellent sliding property. In contrast, PEEK is classified into superengineering plastics presenting highest performance. PEEK is particularly excellent in heat resistance and chemical resistance among other super engineering plastics and is known as a very reliable resin.

PEEK that is a constituent of rotary blade unit 111e is larger in strength and wear resistance than polyacetal constituting bearing unit 111a. Polyacetal constituting bearing unit 111a is self-lubricating, and has a low coefficient of friction with metal, in particular. Shaft unit 110 is internally formed of metal such as a stainless steel material (e.g., SUS316). Polyacetal constituting bearing unit 111a is higher in slidability for metal than the PEEK constituting rotary blade unit 111e and is thus suitable for a bearing member.

As shown in FIGS. 9 and 10, agitator 111 has bearing unit 111a inserted into hole 111f of body 111c formed of PEEK. Bearing unit 111a is disposed rotatably relative to a shaft located inside shaft unit 110 and formed of metal, and has a bottom surface fixed by locking unit 111b.

Agitator 111 has bearing unit 111a composed of polyacetal having a self-lubricating property and a low coefficient of friction with metal. Therefore, no shaving results even if bearing unit 111a slides with shaft unit 110 for a long period of time. Agitator 111 has rotary blade unit 111e composed of PEEK, and even if it rotates for a long period of time, no shaving results between agitator 111 and oxygen intake and discharge pipes 121 and 125 located at a position at which the rotary blade unit comes into contact with the pipes. This allows a reliable analysis of metabolites of microorganisms.

In particular, in the present embodiment, as shown in FIGS. 6 to 8, baffle plate 110b has an end fixed to annular component 110a, and agitator 111 rotates at a position lower than annular component 110a. As a result, baffle plate 110b does not slide with respect to a portion coating rotary blade unit 111e or magnet unit 111d of agitator 111, and shaving can be prevented. This allows a reliable analysis of metabolites of microorganisms.

### Flow of Sampling

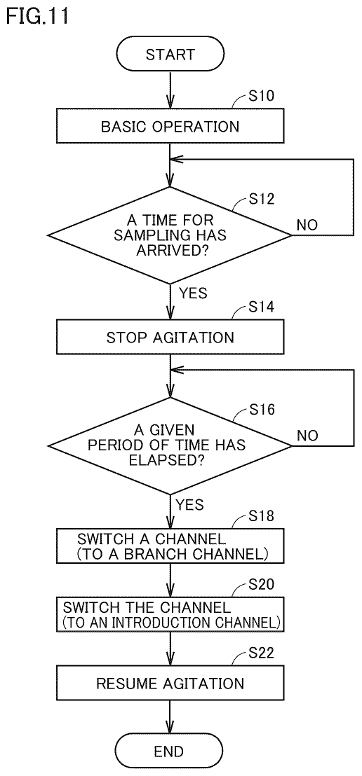

FIG. 11 is a flowchart of a process performed in sampling apparatus 1 to sample a culture solution in cell culturing device 100 and introduce the sampled culture solution into test tube 14. In one implementation, sampling apparatus 1 performs the process of FIG. 11 by the control device 60 CPU 61 executing a given program.

Control device 60 is an example of a controller that controls an operation of an agitator (agitator 111) and that of a channel switching unit (valve 22). Control device 60 controls the operation of agitator 111 by controlling an operation of motor 13.

The program may be stored in memory 62. In that case, memory 62 is an example of a storage medium that temporarily stores the program. The program may be stored in a storage medium accessible by CPU 61 and detachably attachable to control device 60. In that case, the storage medium is an example of a storage medium that temporarily stores the program.

The process of FIG. 11 is performed for each test tube 14. In the following, for convenience of explanation, sampling from the leftmost one of the three cell culturing devices 100 shown in FIG. 2 will be described. That is, in this description, channel 41 constitutes the first circulation channel together with extraction channel 43 and introduction channel 44. The flow of the process will now be described with reference to FIG. 11.

In step S10, control device 60 causes cell culturing device 100 to perform a basic operation. The basic operation includes circulating a culture solution through the first circulation channel and circulating a reagent through the second circulation channel. Circulating the culture solution through the first circulation channel includes rotating motor 13 to rotate agitator 111 to agitate the culture solution in cell culturing device 100.

In step S12, control device 60 determines whether a time to sample the culture solution and introduce it into test tube 14 has arrived. Until control device 60 determines that the time has arrived, the control device repeats the determination in step S12 (NO in step S12). When control device 60 determines that the time has arrived (YES in step S12), the control device proceeds with step S14.

In step S14, control device 60 stops the rotation of motor 13 to stop the rotation of agitator 111. This stops the agitation of the culture solution in cell culturing device 100.

In step S16, control device 60 determines whether a given period of time has elapsed since the agitation was stopped in step S14. Until control device 60 determines that the given period of time has elapsed, the control device continues the control in step S16 (NO in step S16), and once the control device has determined that the given period of time has elapsed (YES in step S16), the control device proceeds with step S18.

In step S18, control device 60 causes valve 22 to switch a channel so that a liquid in channel 41 is guided to branch channel 42.

In step S20, after a specific period of time has elapsed since control device 60 caused valve 22 to switch a channel in step S18, control device 60 causes valve 22 to switch the channel so that the liquid in channel 41 is guided to introduction channel 44. The specific period of time in step S20 means a period of time corresponding to a given amount of sampling.

In step S22, control device 60 resumes rotating motor 13 to resume agitating the culture solution in cell culturing device 100. Subsequently, control device 60 ends the process of FIG. 11.

In the process described above with reference to FIG. 11, in step S18, a channel is switched by valve 22 for sampling and introducing a culture solution into test tube 14. Note that agitation in cell culturing device 100 is stopped a given period of time before the channel is switched in step S18 (step S14). That is, the agitation is stopped in step S14, and thereafter once the given period of time has elapsed, the channel is switched in step S18 to start sampling.

Sampling after the agitation is stopped in cell culturing device 100 for “a given period of time” minimizes variation between an amount of gas dissolved in a sampled culture solution for one sampling and an amount of gas dissolved in a sampled culture solution for another sampling. An excessively short “given period of time” results in insufficiently suppressed variation in the amount of dissolved gas for each sampling. On the other hand, an excessively long “given period of time” is expected to result in uneven culturing in the sampled culture solution for each sampling. In this sense, in one implementation, the “given period of time” may be set between 2 minutes and 10 minutes. In another implementation, the “given period of time” may be set between 3 minutes and 7 minutes.

It is to be noted that the agitation may be incompletely stopped insofar as variation in the amount of dissolved gas for each sampling is suppressed. That is, control device 60 in step S14 may not stop rotating agitator 111, and may instead reduce a rotation speed of agitator 111 to be lower than that in the basic operation in step S10. Control device 60 reduces the rotation speed of agitator 111 by reducing that of motor 13. The rotation speed of agitator 111 in step S10 is also referred to as a “basic speed” and is a speed set for circulating the culture solution.

In one implementation, control device 60 in step S14 reduces the rotation speed of agitator 111 to about 1/10 of the basic speed. In that case, after the sampling is completed and a channel is switched in step S20, control device 60 returns the rotation speed of agitator 111 to the rotation speed in step S10.

### Equalization of Amount of Introduction into Test Tube

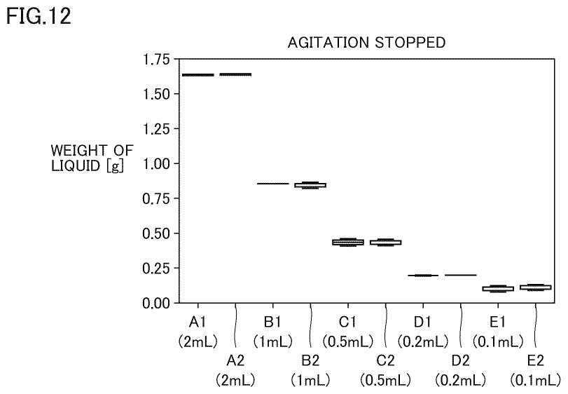

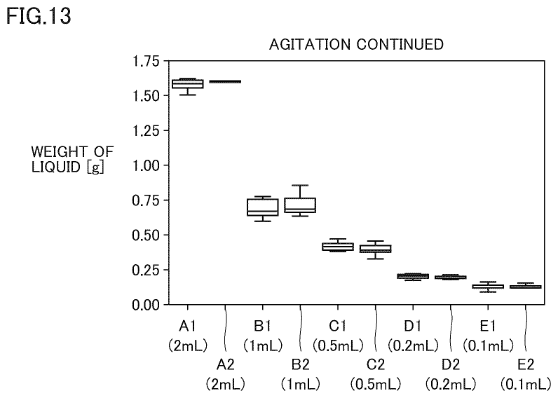

FIG. 12 is a diagram showing an example of a result of an amount of a liquid introduced according to the process of FIG. 11. FIG. 13 is a diagram showing an example of a result of an amount of the liquid introduced according to a comparative example. The results of FIGS. 12 and 13 are obtained when pure water is used as the liquid introduced from cell culturing device 100 into test tube 14. In the graphs of FIGS. 12 and 13, the vertical axis represents the weight of the liquid introduced into test tube 14.

The result shown in FIG. 12 is a result obtained when the agitation by agitator 111 is stopped a given period of time before a liquid is introduced from cell culturing device 100 into test tube 14, as has been described with reference to FIG. 11. In contrast, the result shown in FIG. 13 is a result obtained when the agitation of agitator 111 is continued before and after the liquid is introduced from cell culturing device 100 into test tube 14.

FIGS. 12 and 13 both show for each of ten groups (A1 to E2) a range (or a maximum value and a minimum value) of an amount of the liquid introduced for each of 6 times. Note that a different target volume for the liquid to be introduced is set for each group. A target volume of 2 mL is set to be introduced for groups A1 and A2, a target volume of 1 mL is set to be introduced for groups B1 and B2, a target volume of 0.5 mL is set to be introduced for groups C1 and C2, a target volume of 0.2 mL is set to be introduced for groups D1 and D2, and a target volume of 0.1 mL is set to be introduced for groups E1 and E2.

The result of FIG. 13 shows that any group has a larger difference between a maximum value and a minimum value in weight of the liquid introduced into test tube 14 than the result of FIG. 12.

For example, in FIG. 13, group A1 has a maximum value of 1.65 g and a minimum value of 1.50 g, and a difference therebetween of 0.15 g. With a median value of 1.575 g, the difference between the maximum value and the minimum value, i.e., 0.15 g, is a relatively large value of about 10% with respect to the median value of 1.575 g.

Further, in FIG. 13, group B2 has a maximum value of 0.90 g and a minimum value of 0.60 g, and a difference therebetween of 0.30 g. With a median value was 0.75 g, the difference between the maximum value and the minimum value, i.e., 0.30 g, is a relatively large value of about 43% with respect to the median value of 0.75 g.

In contrast, the result of FIG. 12 shows that any group has a small difference between a maximum value and a minimum value in weight of the liquid introduced into test tube 14. In other words, according to the result of FIG. 12, no group exhibits a substantial variation in weight of the liquid introduced into test tube 14. That is, as has been described with reference to FIG. 11, stopping the agitation by agitator 111 a given period of time before a liquid is introduced from cell culturing device 100 into test tube 14 suppresses variation in a proportion of gas in the solution when it is aspirated, and can thus control the aspirated solution in accuracy. Such an effect can be expected not only when the agitation by agitator 111 is completely stopped, but also when a rotation speed of agitator 111 for agitation is reduced.

### Aspects

It is understood by those skilled in the art that the above-described exemplary embodiments are specific examples of the following aspects:
(Clause 1) A cell culturing device according to one aspect is a cell culturing device that cultures cells, and comprises: a vessel that accommodates a culture solution containing a medium and cells; an agitator that is provided in the vessel and agitates the culture solution; a discharge pipe that discharges the culture solution in the vessel to outside the vessel; and an oxygen intake pipe that draws oxygen into the vessel. The discharge pipe has an end below the agitator in a vertical direction.

The cell culturing device of clause 1 has the discharge pipe below the agitator, and while the culture solution is agitated by the agitator the culture solution can be discharged outside the vessel at a position hardly affected by oxygen bubbling caused by supplying oxygen. The cell culturing device of clause 1 can thus accurately aspirate and dispense a sample while keeping a constant amount of gas dissolved in the culture solution.
(Clause 2) The oxygen intake pipe has an end positioned so as to overlap the agitator in the vertical direction. The cell culturing device of clause 2 has the oxygen intake pipe with an end positioned so as to overlap the agitator. The cell culturing device of clause 2 allows the discharge pipe having an end below the agitator to be less affected by oxygen bubbling caused by oxygen supplied from the oxygen intake pipe.
(Clause 3) A drive device that uses magnetic force to rotatably drive the agitator contactlessly from outside the vessel, and a control device that controls an operation of the drive device are further comprised. After the control device stops the drive device, the culture solution is discharged through the discharge pipe to outside the vessel.

The cell culturing device of clause 3 discharges the culture solution to outside the vessel after the control device stops the drive device. The cell culturing device of clause 3 can aspirate and dispense the culture solution with oxygen bubbling settled, and can thus accurately aspirate and dispense a sample while maintaining a constant amount of gas dissolved in the culture solution.

It should be understood that the presently disclosed embodiments are illustrative and non-restrictive in any respect. The scope of the present disclosure is defined by the terms of the claims, rather than the above description of the embodiments, and is intended to encompass any modifications within the meaning and scope equivalent to the terms of the claims.

### REFERENCE SIGNS LIST

1 sampling apparatus, 2 pretreatment apparatus, 3 liquid chromatograph mass spectrometer, 4 centrifugal separation mechanism, 5 liquid removal mechanism, 6 reagent supply mechanism, 7 agitation mechanism, 8 extraction mechanism, 10 pretreatment system, 12 holding unit, 13 motor, 14 test tube, 20 culture solution sampling mechanism, 21, 31 pump, 22, 23, 32, 33 valve, 25 filter, 26 cleaning liquid tank, 27 waste liquid tank, 30 reagent sampling mechanism, 34 reagent tank, 41, 42, 49, 50 channel, 43, 45, 47 extraction channel, 44, 46, 48 introduction channel, 60 control device, 61 CPU, 62 memory, 100 cell culturing device, 101 vessel, 102 lid unit, 103 DO sensor, 104 pH sensor, 105 cap, 110 shaft unit, 110a annular component, 110b baffle plate, 110c pedestal unit, 111 agitator, 111a bearing unit, 111b locking unit, 111c body, 111d magnet unit, 111e rotary blade unit, 121 oxygen intake pipe, 121a, 125a end, 122 oxygen exhaust pipe, 123 sample adding pipe, 124 suction pipe, 125 discharge pipe.

1. A cell culturing device that cultures cells, comprising:
a vessel that accommodates a culture solution containing a medium and cells; an agitator that is provided in the vessel and agitates the culture solution; a discharge pipe that discharges the culture solution in the vessel to outside the vessel; and an oxygen intake pipe that draws oxygen into the vessel, the discharge pipe having an end below the agitator in a vertical direction.

2. The cell culturing device according to claim 1, wherein the oxygen intake pipe has an end positioned so as to overlap the agitator in the vertical direction.

3. The cell culturing device according to claim 1, further comprising: a drive device that uses magnetic force to rotatably drive the agitator contactlessly from outside the vessel; and a control device that controls an operation of the drive device, wherein after the control device stops the drive device, the culture solution is discharged through the discharge pipe to outside the vessel.
